(19) United States
(12) Patent Application Publication
(10) Pub. No.: US 2020/0245950 A1
(43) Pub. Date: Aug. 6, 2020

(54) REAL-TIME BODY TEMPERATURE MANAGEMENT

(71) Applicant: Flotherm, Inc., Los Angeles, CA (US)

(72) Inventors: Bradley C. LIANG, Bloomfield Hills, MI (US); Abhinav RAMANI, Los Angeles, CA (US); Brian T. KANNARD, Los Angeles, CA (US); Peter Luke SANTA MARIA, Redwood City, CA (US)

(21) Appl. No.: 16/777,895

(22) Filed: Jan. 31, 2020

Related U.S. Application Data
(60) Provisional application No. 62/799,507, filed on Jan. 31, 2019.

(51) Int. Cl. A61B 5/00; G16H 50/30; G16H 10/60; G16H 50/70; A61B 5/01

(52) U.S. Cl. CPC A61B 5/7275; G16H 50/30; G16H 10/60; A61B 2562/0271; A61B 5/01; A61B 5/6828; G16H 50/70

(57) ABSTRACT

Determining a patient's risk of hypothermia involves receiving a first input from a sleeve administered to a patient, receiving a second input, determine a first risk value based at least in part on the first input, determining a second risk value based at least in part on the second input, determining a first relative risk value of the first risk value based at least in part on comparing the first risk value to the second risk value, determining a second relative risk value of the second risk value based at least in part on comparing the first risk value to the second risk value, and performing a risk calculation to generate a risk score for the patient.

### RELATED APPLICATION

This application claims priority to U.S. Provisional Application No. 62/799,507, filed on Jan. 31, 2019, entitled REAL-TIME ASSESSMENT AND REGULATION OF CORE BODY TEMPERATURE, the disclosure of which is hereby incorporated by reference in its entirety.

### BACKGROUND

### Field

The present application relates to medical devices and methods. More specifically, the application relates to methods, devices and systems for regulating body temperature of a mammal.

### Description of Related Art

Each year, over 60 million surgical procedures are performed in the United States. Patient temperatures can drop precipitously during surgery, due to the effects of general anesthesia, lack of insulating clothing, and exposure to cold operating room temperatures.

### SUMMARY

Described herein are one or more methods and devices for managing temperature of a patient, such as a surgical patient, using one or more wearable devices configured to provide heating and/or blood flow augmentation functionality.

Some implementations of the present disclosure involve a method of assessing a patient's risk of hypothermia. The method comprises receiving a first input from a sleeve administered to a patient, receiving a second input, determining a first risk value based at least in part on the first input, determining a second risk value based at least in part on the second input, determining a first relative risk value of the first risk value based at least in part on comparing the first risk value to the second risk value, determining a second relative risk value of the second risk value based at least in part on comparing the first risk value to the second risk value, and generating a risk score for the patient.

The first input may be one of a group comprising core temperature data for the patient, peripheral temperature data, and vital signal data for the patient. In some embodiments, the second input is one of a group comprising demographic data for the patient, comorbidity data for the patient, pharmalogical data for the patient, procedural data relating to a procedure involving the patient, core temperature data for the patient, peripheral temperature data, environmental data, and vital signal data for the patient. The method may further comprise assigning a first weight value to the first input and assigning a second weight value to the second input. Determining the first relative risk value may involve comparing the first weight value to the second weight value. In some embodiments, the method further comprises adjusting a temperature of the sleeve based at least in part on the risk score. The method may further comprise computing a rate of core temperature change value based at least in part on the first input. In some embodiments, the method further comprises determining a core temperature prediction for the patient based at least in part on the rate of core temperature change value. The method may further comprise adjusting a temperature of the sleeve based at least in part on the risk score.

Some implementations of the present disclosure relate to a method comprising determining a set point core temperature value, measuring a present core temperature value of a patient being treated with a sleeve comprising one or more heating elements, comparing the set point core temperature value to the present core temperature value, in response to determining that the present value is not less than the set point value, comparing the present value to a sum of the set point value and a buffer value, in response to determining that the present value is not greater than the sum, maintaining a temperature setting at a first heating element of the sleeve, and in response to determining that the present value is greater than the sum, decreasing the temperature setting at the first heating element of the sleeve.

In some embodiments, the method further comprises, in response to determining that the present value is less than the set point value, measuring a heating element temperature of the first heating element and comparing the heating element temperature to a safety threshold value. The method may further comprise, in response to determining that the heating element temperature is not greater than the safety threshold value, increasing the heating element temperature. In some embodiments, the method further comprises, in response to determining that the heating element temperature is greater than the safety threshold value, comparing the heating element temperature to a maximum temperature value. The method may further comprise, in response to determining that the heating element temperature is not greater than the maximum temperature value, increasing the heating element temperature. In some embodiments, the method further comprises, in response to determining that the heating element temperature is greater than the maximum temperature value, decreasing the heating element temperature. The method may further comprise, in response to determining that the heating element temperature is greater than the maximum temperature value, comparing a compression frequency of a first compression element at the sleeve to a maximum frequency value. In some embodiments, the method further comprises, in response to determining that the compression frequency is not greater than the maximum frequency value, increasing the compression frequency. The method may further comprise, in response to determining that the compression frequency is greater than the maximum frequency value, maintaining the compression frequency.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 provides front and side views of a sleeve configured to provide blood flow and/or compression therapy to a patient in accordance with some embodiments.

FIG. 2 provides a view of a sleeve configured to provide heating and/or compression via a first portion configured to contact a calf of a patient and a second portion configured to contact a sole of a foot of the patient in accordance with some embodiments.

FIGS. 3A and 3B illustrate systems including one or more cable components connecting a sleeve to a controller in accordance with one or more embodiments.

FIG. 4 provides an example risk-weighted self-adjusting calculation process for determining a patient's hypothermia risk in accordance with some embodiments.

FIG. 5 illustrates a process for predicting a patient's future body temperature value given a raw/primary data input in accordance with some embodiments.

FIG. 6 provides a graph illustrating temperature values at different moments of time in accordance with some embodiments.

FIG. 7 provides graphs illustrating venous flow velocity over time in accordance with some embodiments.

FIGS. 8A and 8B illustrate on-off cycles which may be performed at a controller for one or more sleeves configured to warm and/or compress one or more target areas of a patient's body in accordance with some embodiments.

FIG. 9 provides a process for enabling heating elements at sleeve devices providing heat to a patient in accordance with some embodiments.

FIG. 10 provides a process for increasing temperature at heating elements at sleeve devices providing heat to a patient in accordance with some embodiments.

FIG. 11 illustrates a process for controlling a heating element of a sleeve in accordance with some embodiments.

FIG. 12 provides a process for increasing and/or maintaining patient temperature using a sleeve configured to provide heat and/or compression to a patient in accordance with some embodiments.

### DETAILED DESCRIPTION

The headings provided herein are for convenience only and do not necessarily affect the scope or meaning of the claimed invention.

### Overview

Each year, over 60 million surgical procedures are performed in the United States. While great care may be taken to prevent surgical complications, one commonly overlooked and under-addressed problem is the risk of developing hypothermia before, during, or after surgery (referred to as “inadvertent perioperative hypothermia” or “IPH”). Patient temperatures can drop precipitously during surgery due to the effects of general anesthesia, lack of insulating clothing, and exposure to cold operating room temperatures. Even with today's standard of care, 30-50% of surgical patients may develop hypothermia.

Hypothermia often causes much more than patient discomfort. Patients who suffer even mild IPH can face a significantly elevated risk of developing surgical site infections, cardiac morbidities, intraoperative bleeding, and other avoidable complications. Together, these complications can significantly increase recovery time and overall length of hospital stay, leading to increased costs for all parties. By some estimates, the unmanaged risk for IPH is a $15 billion problem in the United States alone, and yet it is largely overlooked.

Perioperative heat loss can occur predominantly via convective heat transfer, particularly through the palms of the hands, soles of the feet, and exposed surgical site surface area. During preoperative care, patients are often dressed solely in a gown and are often exposed to relatively cold waiting areas with little to no insulation. Although patients are generally only anesthetized at the start of surgery, patients often arrive at the surgical theater moderately hypothermic. This can put a patient at greater risk for developing severe hypothermia once anesthesia has been administered. Postoperative drops in core temperature can increase the likelihood of developing additional comorbidities, such as morbid cardiac outcomes, surgical site infections, and blood loss, any of which can prolong recovery and hospitalization.

Patients undergoing surgery can develop hypothermia during the surgical procedure itself, especially when the procedure involves the patient's core area, such as procedures involving the posterior or anterior sides of the thoracic, abdominal, and pelvic regions. Surgeries of the core involve the exposure of vital internal organs to the colder environment and thus carry a greater risk of hypothermia. Furthermore, core surgeries often necessitate uncovering of the trunk and chest, which render blankets and many other currently-available interventions inadequate. Once in the operating room, patients may be naked and exposed to a room temperature well below 36 degrees Celsius and to cold liquids used to wash the surgical site during sterilization preparation. At the onset of surgery, delivered anesthetics can immediately impair the normal autonomic thermoregulatory controls. Colder blood may be transferred from the peripheries of the body to the core through a phenomenon known as redistributive hypothermia. Vasodilatation and reduction in muscle tone can cause a significant drop in core temperature within the first half hour of surgery.

Overall, compared to non-hypothermic patients, those who suffer from IPH experience greater rates of surgical site infections, bleeding, and cardiac complications. Such issues may require additional monitoring and/or increase the length of stay and/or subjective discomfort. The development of IPH is strongly correlated with a multitude of physiological organ system changes impacting the cardiovascular, respiratory, neurologic, immunologic, hematologic, drug-metabolic, and wound-healing mechanisms. The incidence of several post-surgical complications can be increased due to even mild hypothermia.

Intraoperatively, hypothermia can cause a decrease in cardiac output and heart rate, which can lead to ventricular dysrhythmias. Platelet functions can become impaired and there can be a decrease in coagulation factors, which can in turn lead to greater intraoperative bleeding and blood loss. Impaired immune functions can increase the rate of surgical site infections. Hypothermia is associated with a four-fold increase in surgical wound infection and twice as many morbid cardiac events. In select procedures such as colorectal, gynecologic, or spinal surgery, where infection rates are normally higher than other surgeries, hypothermia can be exceedingly dangerous to the intraoperative and postoperative recovery. These complications and others are supported in multiple studies and can result in both clinical and economic burdens.

Current methods of preventing hypothermia may not be completely effective. Even with the current interventions, up to 46% of patients are reported to be hypothermic at the start of surgery, and 33% are hypothermic upon arrival to the post-anesthesia care unit (PACU). Assuming the cost savings for maintaining normothermia in one patient is approximately $5,000 per patient, and approximately 30% of the 17 million high-risk surgical patients are hypothermic, a system-wide cost savings of $15 billion could be realized by keeping these patients normothermic. With rising healthcare costs and recent initiatives mandating the maintenance of perioperative normothermia, hospital administrators nationally are in need of new, efficacious and cost-effective devices to address perioperative hypothermia, a product space which has seen little innovation since the introduction of the forced air warming blanket nearly three decades ago.

Some devices for perioperative warming may include forced-air temperature-management devices (e.g., warming blankets). Some temperature-management solutions utilize high-heat transfer conduction heating blankets and intraoperative hand-warming devices. However, such solutions can be associated with various key shortcomings including, for example: (1) undesirably high risk of contaminating the surgical field (e.g., forced-air methods can blow bacteria-containing air into the surgical field); (2) forced-air devices can get in the way (e.g., to warm the core, forced-air blankets may need to be in contact with the core, which may be near to the surgical site); and (3) operating room staff may turn down the temperature on a device due to their own comfort (e.g., staff members may turn down the patient's forced-air device due to the device heating the surrounding air). Moreover, certain devices may not be used in preoperative warming for one or more of the following reasons, among others: (1) some devices may immobilize the upper limbs, impeding patient mobilization; (2) devices may be cumbersome (e.g., a device may float on the patient and get blown off or fall off during use and/or transport, and they require large, predominantly floor-based blowers that may not be mobile; (3) they may not attach to the patient and/or can become dislodged during transport and obstruct the bed and other monitors and devices; and (4) they can require a conscious administrative decision to implement.

Embodiments of the present disclosure advantageously provide certain improved methods and systems for maintaining a patient's core body temperature before, during, and/or after surgery. Furthermore, embodiments described herein provide methods and systems for core body temperature-management in an unobtrusive, effective, and easy-to-use (e.g., easy to set-up) manner. Some embodiments of the present disclosure can be suitable for use before, during, and/or after a surgical procedure and can be acceptable to the patient while awake in the preoperative and/or postoperative settings. Some devices, methods, and systems herein advantageously provide for at least partially automated management of patient temperature, limiting the need for clinician input in maintaining patient target temperatures. For example, embodiments of the present disclosure advantageously provide closed-loop temperature-management solutions.

Closed-loop temperature-management may involve at least partially automated adjustment of heat transfer to the body in response to real-time measurement of patient temperature. The automated regulation of heat delivered to a patient may be suitable to improve temperature control through elimination of manual errors and/or improved efficiency (e.g. reduction in time required to adjust therapy). Methods, devices, and systems implementing or relating to the various temperature control determinations and processes disclosed herein for providing therapy automation can greatly reduce and/or potentially eliminate the need for certain types of clinician input and oversight in adjusting temperature and/or blood flow therapy settings/parameters and are well suited towards maintenance of patient temperature at a predetermined set point/value (or within a defined range) throughout the perioperative timeframe.

In some implementations, the present disclosure relates to devices, systems and methods directed toward automated application of warming and blood flow (WBF) therapy to a patient to help regulate body temperature, reduce blood stasis, deep vein thrombosis, pulmonary emboli, and/or optimize blood circulation. WBF therapy can be implemented in the systems, devices and methods described herein to dually increase patient temperature and improve circulation to the body's core from one or more extremities. Patient warming may be accomplished in several different ways, including but not limited to the conductive application of heat to areas on the skin surface of the body. Increased blood circulation may be accomplished in several different ways, including but not limited to intermittent compression, such as in the area of the patient's calf.

In some implementations, the present disclosure relates to systems, devices, and methods for determining patient risk for hypothermia in real-time in response to multiple inputs, including but not limited to core temperature measurement, anesthesia onset (e.g., timestamp), and/or patient demographic information (e.g., age, sex, weight, etc.). WBF and intermittent compression therapy delivery can be modulated by the system/device(s) in response to patient risk for, detection of, and/or prediction of, oncoming hypothermia.

In some embodiments, systems, devices, and/or methods to enable the real-time determination of patient risk of developing hypothermia are provided. Such systems/devices can include one or more sensors or sensor arrays for continuous monitoring of patient peripheral temperature. Systems/devices of the present disclosure may further comprise one or more electronics modules/controllers configured to power and/or communicate with the sensor(s). Such electronics modules/controllers can include certain control circuitry, includes one or more processors and/or memory/data storage devices that may be configured to determine risk of patient hypothermia based on at least one temperature input, which may advantageously be continuously, periodically, and/or sporadically monitored. In some embodiments, the systems/devices may include one or more electronic visual display devices, interfaces, lights, or other type of visual output for indicating relevant patient metrics (e.g. hypothermia risk). The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

In some implementations, the present disclosure relates to systems, devices, and methods involving the application of a physiological heat transfer model to estimate patient tissue and core temperatures, derived from certain available input(s), including but not limited to, for example, peripheral temperature readings.

Some embodiments further utilize one or more sets of primary inputs including but not limited to medical procedural parameters, time-to and -from induction of anesthesia, and patient temperature readings (e.g., actual or estimated core temperature). Some embodiments further include secondary inputs including, for example, information available from a patient's electronic health record (e.g. demographics, comorbidities, pharmacological agents) or other physiological (e.g. vital signs) or environmental (e.g. room temperature) monitors/parameters.

In some implementations, the present disclosure relates to systems, devices, and methods may include a controller comprising certain control circuitry configured to adjust WBF therapy parameters and/or control operations to maintain normal core body temperatures. In some embodiments, the system controller and/or associated control circuitry may be configured to adjust therapy parameters and/or control operations based at least in part on a determined patient risk level/value of developing hypothermia. In some embodiments, therapy adjustments made by the system/device(s) may be applied dynamically over time.

Although this invention has been described in more detail below, the scope of the invention as set forth in the following description should not be limited by the foregoing descriptions of various embodiments. Thus, it is intended that the scope of the present invention herein disclosed should not be limited by the particular disclosed embodiments, but should be determined only by a fair reading of the content presented herein/herewith.

### Temperature-Management Systems

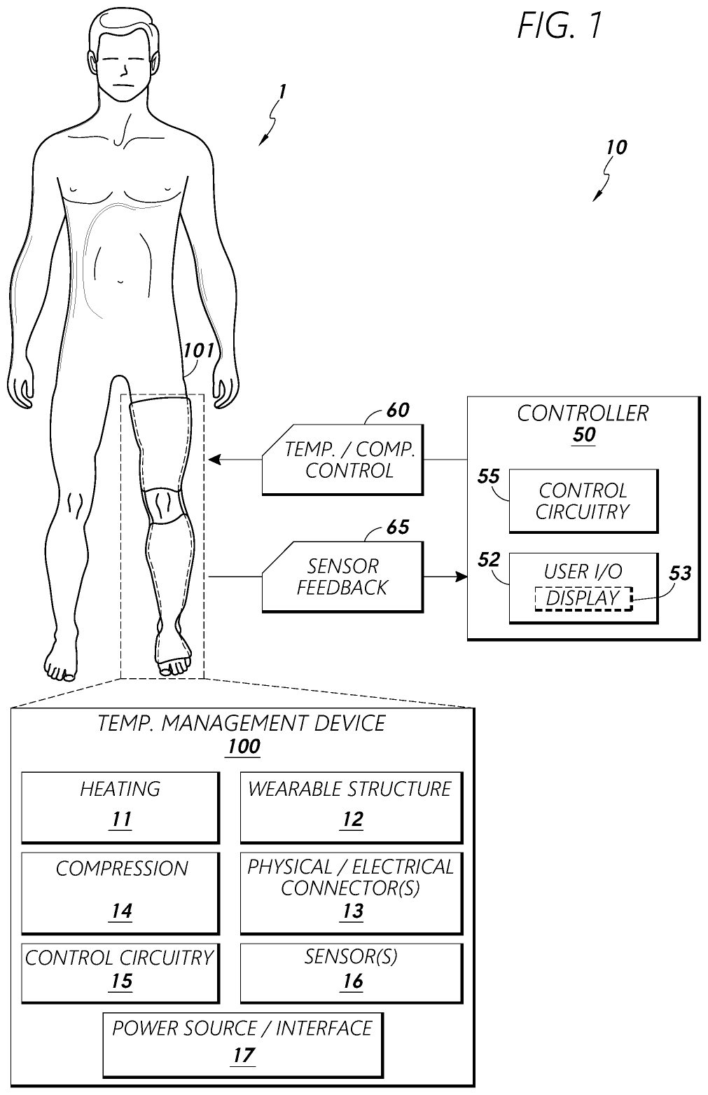

Disclosed solutions for managing temperature of a patient may be implemented in connection with a temperature-management system. FIG. 1 illustrates a system 10 for managing core temperature of a patient 1 in accordance with embodiments of the present disclosure. Although the description of FIG. 1 and other embodiments herein is generally presented in the context of temperature management, it should be understood that description of temperature-management and control herein is applicable to blood flow management solutions as well.

FIG. 1 shows a system 10 for managing temperature and/or blood flow in a patient 1 according to one or more embodiments. The patient 1 can have a temperature-management device 100 disposed on, for example, a limb and/or associated anatomy, of the patient 1. For example, the temperature-management device 100 can be disposed at least partially on the patient's leg 101. The temperature-management device 100 can include one or more sensors/sensor transducers 16, such as one or more microelectromechanical system (MEMS) devices, such as MEMS temperature sensors, or the like.

The system 10 can be used to deliver warming therapy and/or blood flow therapy to the patient 1 to help reduce blood stasis, deep vein thrombosis, and/or pulmonary emboli and/or to help regulate body temperature and/or optimize blood circulation. Warming and/or blood flow therapy can be used in the system 10 to help maintain normothermia and/or help return circulation to the patient's core, including the heart and lungs, from one or more extremities/limbs, such as the leg 101. Blood flow therapy and/or blood circulation therapy may be accomplished in a number of different ways, including but not limited to intermittent compression. For example, in some implementations, intermittent compression may be performed through the execution of circumferential compression of one or more limbs. Warming therapy may likewise be accomplished in a variety of different ways, including without limitation through the use of ultrasound, electrical, mechanical, chemical, radiative and/or convective energy.

The temperature-management device 100 may have any suitable or desirable shape, form, and/or configuration. For example, FIG. 1 provides a front view of an example temperature management sleeve device 100 configured to provide blood flow and/or compression therapy to a patient, which may represent an embodiment of a temperature-management device that may be used in connection with any of the embodiments disclosed herein. The term “sleeve” is used herein according to its plain and ordinary meaning and may refer to any device configured to be administered to one or more areas of a human body for delivery of heat and/or compression to the human body. For example, a “sleeve” may be a device configured to provide therapy to a limb or other body part at least in part through physical contact with the skin and/or other feature(s) of the body, wherein such physical contact provides therapy in and of itself or facilitates the provision of therapy through physically securing, positioning, or otherwise arranging one or more therapeutic devices, components, or features coupled to or otherwise associated with the sleeve. In some embodiments, the sleeve 100 may comprise a single continuous form or device and/or may be configured to apply therapy to a patient's thigh, knee, calf, and/or foot, and/or one or more other lower limb portions of a patient's body. The sleeve 100 may be applied to a patient's limb 101 (e.g., a leg, arm, and/or foot) and/or may be configured to deliver warming and/or to apply blood flow therapy to at least one area of the patient's limb 101. In some embodiments, the sleeve 100 may be configured to deliver heat to a majority of, or even the entire, limb 101 in conjunction with blood flow therapy. In some embodiments, the sleeve 100 may be configured to deliver heat to at least two different areas on the limb 101 while applying blood flow therapy between, adjacent to, and/or or overlapping the same areas.

In certain embodiments, the managing system 10 can comprise at least two subsystems, including a wearable subsystem or device 100 that includes the sensor(s) 16 (e.g., temperature sensor(s)), as well as control circuitry 15 comprising one or more microcontroller(s), discrete electronic component(s), and one or more power and/or data transmitter(s) (e.g., antennae). The temperature-management system 10 can further include a control subsystem including a controller module/device 50. The controller 50 may be configured to communicate data and/or power with the device 100 in any suitable or desirable manner, such as over a wired or wireless connection. For example, the control circuitry 550 may include certain connectivity circuitry including possibly a wireless transceiver that is electrically and/or communicatively coupled to the control circuitry 15 of the device 100

In some embodiments, the temperature-management device 100 comprises one or more heating elements or mechanisms 11 (e.g., convective and/or conductive/radiative heating mechanism(s)), one or more flood-flow-inducing compression devices or mechanisms 14 (e.g., inflatable bladder(s)), one or more temperature sensors 16 (e.g., thermistors, surface temperature sensors, etc.) integrated with a functional wearable sleeve structure 12 including one or more sleeve portions. The temperature-management device 100 may further include one or more power sources or interfaces 17 as well as one or more electrical connectors for interfacing with a power source, fluid source, data source, and/or the like.

The sensor (s) 16 can comprise one or more MEMS sensors, optical sensors, piezoelectric sensors, electromagnetic sensors, strain sensors/gauges, accelerometers, gyroscopes, and/or other types of sensors, which can be disposed in a manner so as to be positioned on or in proximity to the skin of the patient 1 when the device 100 is worn by the patient 1. The sensor(s) 16 may be associated with the wearable structure 12, such that at least a portion thereof is contained within, or attached to, the wearable structure 12. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. The sensor(s) 16 is/are electrically and/or communicatively coupled to the control circuitry 15, which may comprise one or more application-specific integrated circuit (ASIC) microcontrollers or chips.

In certain embodiments, the sensor (s) 16 can be configured to generate electrical signals that can be wirelessly transmitted to the controller 50. In order to perform such wireless data transmission, the temperature-management device 100 can include radio frequency (RF) transmission circuitry, such as a signal processing circuitry and an antenna. The control circuitry 15 of the temperature-management device 100 can comprise, for example, one or more chips or dies configured to perform some amount of processing on signals generated and/or transmitted using the device 100. However, due to size, cost, and/or other constraints, the temperature-management device 100 may not include independent processing capability in some embodiments.

In certain embodiments, the control circuitry of the temperature-management device 100 and/or the controller 50 includes some amount of volatile and/or non-volatile data storage. For example, such data storage can comprise solid-state memory utilizing an array of floating-gate transistors, or the like. The control circuitry may utilize data storage for storing sensed data collected over a period of time.

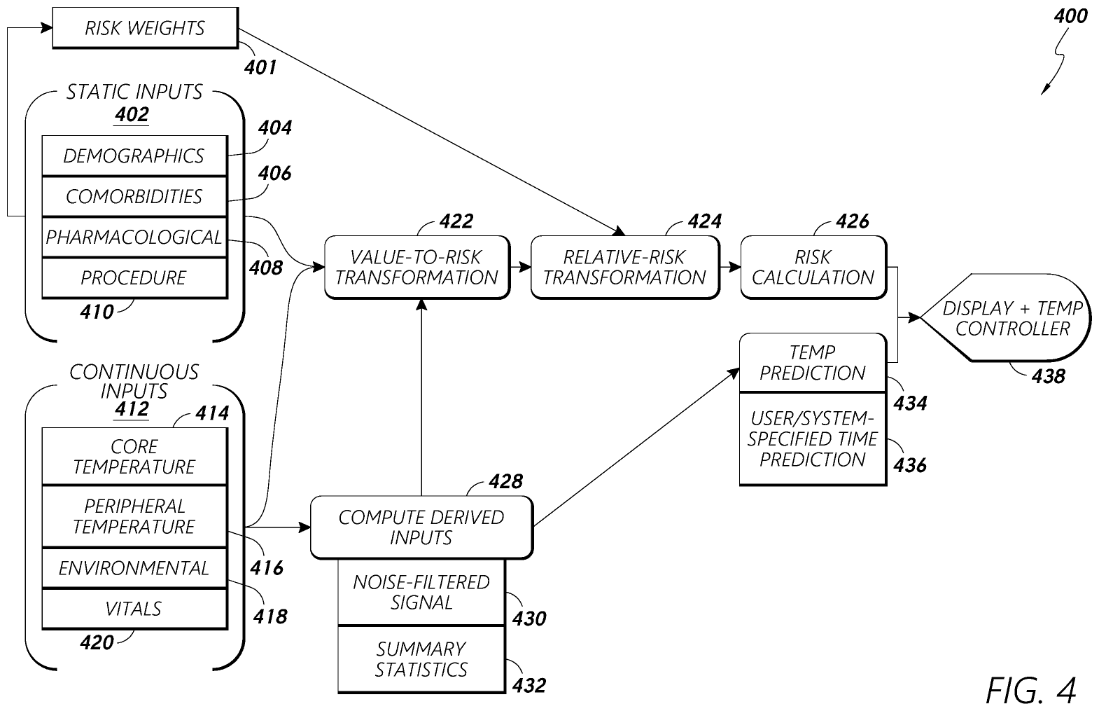

The control circuitry 15 of the temperature-management device 100 may be configured to receive sensor signals from the sensor(s) (e.g., temperature sensor(s)) 16 and transmit sensor feedback data 65 to the controller 50. The controller 50 may in turn utilize the control circuitry 55 to generate certain control signals 60 and provide the same to the temperature-management device 100 to thereby direct operation thereof at least in part. The controller 50 may include certain user input/output (I/O) component(s) 52, such as one or more electronic displays 53, lights, buttons, and/or the like. The control circuitry 15, 55 of either or both of the device 100 and the controller 50 may be configured to implement any of the temperature-management functionality disclosed herein, including with respect to any of the operations, modules, elements, components, and/or other features associated with FIGS. 4-12 and described below. Although the controller 50 is shown in FIG. 1 as separate from the temperature-management device 100, it should be understood that any or all of the components and/or functionality described herein as associated with the controller 50 may be implemented as part of the temperature-management device 100 and any or all of the components and/or functionality described herein as associated with the temperature-management device 100 may be implemented as part of the controller 50. Examples of temperature-management devices and related/associated features that may be implemented in connection with any of the embodiments of the present disclosure are disclosed in U.S. patent application Ser. No. 16/777,894, Filed on Jan. 31, 2020, and entitled PATIENT TEMPERATURE AND BLOOD FLOW MANAGEMENT, the disclosure of which is hereby expressly incorporated by reference and is considered part of the present disclosure.

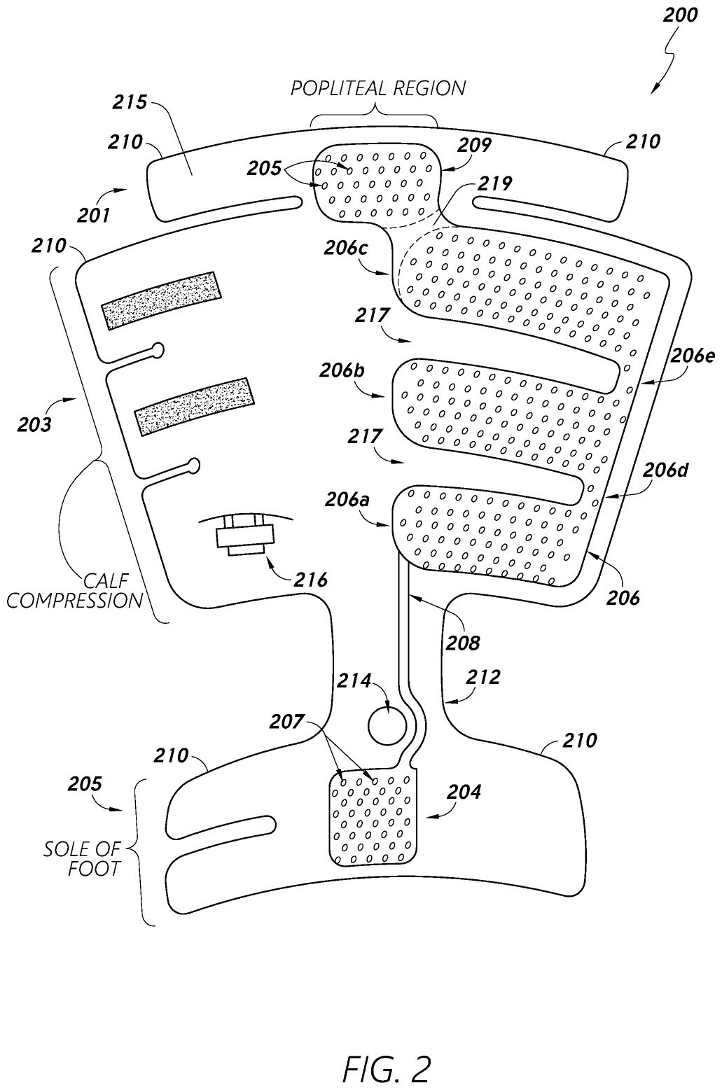

FIG. 2 provides a view of a sleeve 200 configured to provide heating and/or compression via a first portion 201 configured to contact the back of a knee of a patient, a second portion 203 configured to contact a calf of a patient, and/or a third portion 205 configured to contact a foot of the patient. In some embodiments, the sleeve 200 may be configured to provide heat therapy using convective and/or other heating methods. The sleeve 200 may comprise one or more heating and/or compression bladders 206 which may be connected (e.g., in fluid communication) and/or may be separated (e.g., fluidly isolated) from each other. In some embodiments, one or more channels 208 may be configured to provide transport of fluid (e.g., gas) to one or more bladders 206, 204, 209 for delivering heating and/or compression. The one or more bladders 206 may comprise one or more perforations 205 positioned and configured to pass heated air/fluid to targeted areas of the patient's body (e.g., the popliteal region and/or the sole of the foot). Although referenced using separate reference numbers, in some embodiments, two or more of the bladders 204, 206, 209 are in fluid communication with one another.

The sleeve 200 may comprise multiple portions configured to contact and/or provide heat and/or blood flow therapy to one or more areas of a patient's limb. For example, the sleeve 200 may comprise a first portion 201 configured to provide heat and/or compression to a patient's knee (e.g., at the popliteal fossa) and/or thigh, a second portion 203 configured to provide heat and/or compression to a patient's calf and/or surrounding areas, and/or a third portion 205 configured to provide heat and/or compression to a patient's foot (e.g., the sole of the foot) and/or the surrounding areas.

In some embodiments, channels 208 and/or bladders 206 for providing blood flow and/or compression therapy may not have perforations in at least one or more portions thereof. Bladders 206 for compression may utilize flowing air for sequential compression. Bladders (e.g., 204 and/or 209) configured to provide heating may have perforations 207 and/or may be configured to provide a relatively continuous stream of heated air/fluid for compression and/or heating therapy. In some embodiments, skin/tissue contact may be achieved without compression bladders 206. For example, one or more inserts (e.g., foam insert(s)) may be disposed in or on the sleeve 200 to press the bladders 206 and/or the perforations 205 against the patient's skin to maintain contact between the sleeve 200 and the patient's skin at least in certain desired areas. The number and/or size of the perforations 205 can affect compression. For example, air may escape more easily with a greater number and/or size of the perforations 205, thereby affecting the pressure within the sleeve 200.

With respect to the compression bladders 206, in some embodiments, some bladders 206 may not start filling until other bladders 206 reach a certain pressure. For example, fluid may be provided to the bladders 206 through the channel 208, initially passing into the lower/first bladder portion 206a. The first bladder portion 206a may be fluidly coupled to the second/intermediate bladder portion 206b via an interconnection channel 206d. In some embodiments, fluid may not propagate through the channel 206d into the second bladder portion 206b in substantial amounts until the fluid in the first bladder portion 206a reaches a certain pressure level due to the filling of the first bladder portion 206a. That is, the fluid entering the bladder 206a may sequentially fill the first bladder portion 206, then the second bladder portion 206b, and then the upper/third bladder portion 206c (via the interconnecting channel 206e). Although a certain amount of fluid may pass into the second 206b and third 206c bladder portions prior to the first bladder portion 206a reaching a maximum or threshold volume and/or pressure, the degree to which the first bladder portion 206a fills with fluid may be greater initially compared to the other bladder portion(s). Likewise, the second bladder portion 206b may fill to a greater degree and/or more quickly than the third bladder portion 206c prior to the second bladder portion 206b reaching a maximum or threshold volume and/or pressure. The heat-transfer fluid may further pass to the popliteal bladder portion 209. In some embodiments, the popliteal bladder or other type of heating element may be isolated from the bladder portions 206, such as by a break or barrier portion 219. The interconnection channels 206d, 206e may be sized/dimensioned to produce/control desired sequence/timing of sequential filling of the respective bladder portions 206.

In other embodiments, the first bladder portion 206a, second bladder portion 206b, and/or third bladder portion 206c may be independent of other bladder portions 206. For example, the first bladder portion 206a may not be connected to the second bladder portion 206b by a first interconnection channel 206d and/or the second bladder portion 206b may not be connected to the third bladder portion 206c by a second interconnection channel 206e. Moreover, in some embodiments, one or more bladders 206 may be pressure-controlled independently by an individual fluid channel 208. For example, the sleeve 200 may comprise multiple fluid channels 208 in which at least one of the multiple fluid channels 208 may provide pressure control to only one of the bladders 206.

In some embodiments, one or more bladders 206 may have various features to enable easier wrapping of the sleeve 200 around the patient's limb. For example, a bladder 206 may comprise dimples and/or other features. Furthermore, the bladders 206 may be separated by break portions 217.

In some embodiments, one or more channels 208 for delivering heated air and/or fluid may not have perforations 205 and/or may act as bladders that may be configured to inflate/deflate with a single port. Air can be cycled in and out of a heated bladder on a higher frequency than compression bladders 206. For example, if compression bladders 206 are cycled 1-2-3, heated bladders (e.g., 209, 204) may be cycled with each compression cycle 1-1-1. A cycle may have a duration of approximately sixty seconds but may be adjusted depending on an amount of heat dissipation. In some embodiments, the sleeve may comprise a single bladder 206 utilizing intermittent compression.

Compression may be controlled such that whenever heating is active, compression at target heating areas may be maintained. For example, compression at or near the popliteal fossa and/or the foot may be maintained during heating cycles to ensure that the generated heat is transferred to the popliteal fossa and/or foot. Compression bladders 206 may be filled with additional air/fluid when pressure at the compression bladders 206 is detected below a threshold pressure value. In some embodiments, a foam pad may be utilized to compress the heating bladders against the target areas.

Heating may be delivered via a sheet-type heating element/device, which may utilize either a convective or conductive configuration. Compression bladders 206 may be separate from the heating sheet. In some embodiments, the compression bladders 206 may be configured to maintain an ON state in which the compression bladders 206 continuously press inward in the direction of the skin of the patient. In some embodiments, one or more foam pads may be utilized in place of one or more compression bladders 206.

In some embodiments, heating may be delivered at least in part by fluid escaping and/or passing through perforations 205 of the sleeve 200, which may or may not be associated with the compression bladder portions 206 in addition to the heating portions 204, 209. In some embodiments, the sleeve 200 may comprise one or more straps 210 configured to be wrapped at least partially around a knee and/or other portion of a patient's limb. The arms 210 may be adjustable to allow for wrapping around patients of different sizes. For example, the straps 210 may include Velcro or other types of fastening features for fastening the straps 210 to one another around the patient's limb. Moreover, the length of the sleeve 200 may be adjusted (e.g., at a neck portion 212 between the second portion 203 and the third portion 205) by extending and/or tightening portions of the sleeve 200 and/or by folding and/or securing portions of the sleeve 200 onto and/or to other portions of the sleeve 200.

In some embodiments, the second portion 203 may be configured to provide heating and/or compression to the calf of the patient. A single supply or multiple supplies of heated or non-heated fluid may be used to provide heating to the various bladder portions 206 of the sleeve 200.

The sleeve 200 may comprise one or more features configured to enable easier application of the sleeve 200 to patients. For example, the sleeve 200 may comprise a heel locator 214 configured to be positioned at/over the patient's heel. The heel locator 214 may comprise an opening/cavity and/or visual marker in the sleeve 200. In some embodiments, the sleeve 200 may comprise an inlet and/or outlet port 216 configured to receive fluid, gas, and/or electricity from an external source (e.g., a controller) and/or have fluid drawn therefrom. As shown, the port 216 may be accessible outside of the sleeve to allow for engagement therewith using a corresponding connector associated with a fluid and/or electrical supply device.

Like other embodiments of devices described herein, the sleeve 200 may provide various advantages compared to certain alternative temperature management solutions, including ease of application and/or positioning of the devices on patients. Such devices may include various features (e.g., visual and/or physical indicators) for helping users avoid mistakes in application.

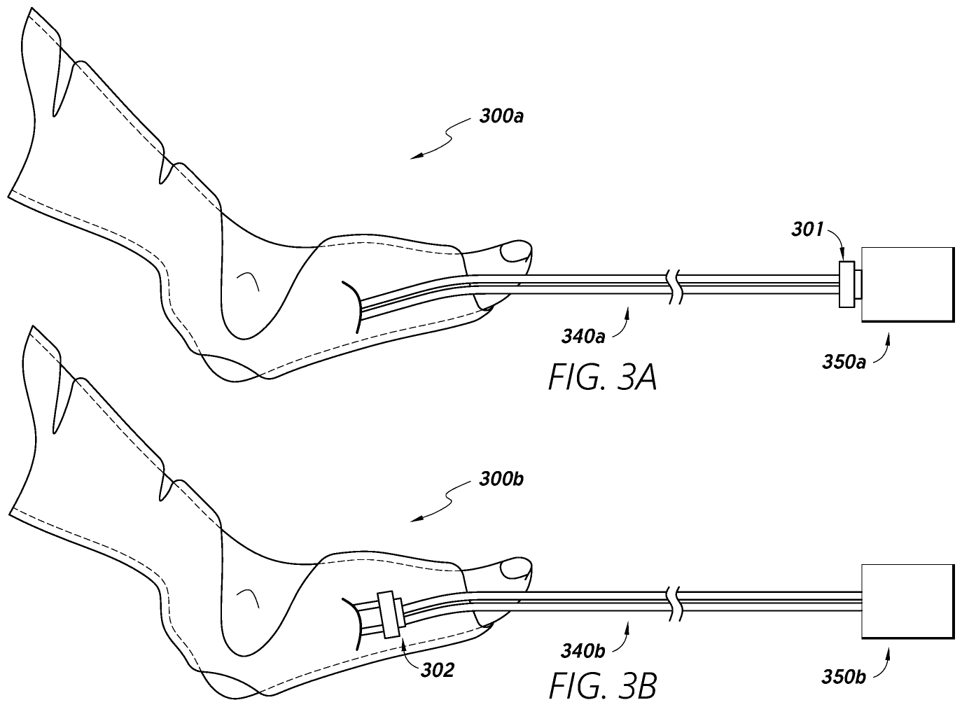

FIGS. 3A and 3B illustrate systems including one or more cables, wires, and/or tubes 340 (referred to individually and/or collectively in the following description as “cable components”) connecting a sleeve 300 to a controller 350. In some embodiments, fluid provided to the sleeve 2000 via the cable component(s) 340 may be heated within the controller 350 to a specified temperature before delivery to the sleeve 300. The fluid may be selectively heated so that only bladders at the sleeve 300 covering specific anatomical regions may be temperature modulated. In some embodiments, temperature control of the warming therapy may be open-loop (e.g., manually specified temperatures) or closed-loop (e.g., automatically controlled to maintain the desired temperature profile, such as in response to a sensor (e.g., temperature, pressure, etc.) feedback). For example, temperature feedback may be generated and/or provided relating to any of esophageal, tympanic, oral, inguinal urinary, and rectal temperatures.

In the illustrated configuration/embodiment of FIG. 3A, the connector 301 is associated with a distal end of the cable 340, which is coupled to or integrated with the sleeve device 300a, whereas in the illustrated configuration/embodiment of FIG. 3B, a connector associated with a distal end of a cable 340b that is coupled to or integrated with the controller 350b is connected to a corresponding connector 302 of the sleeve 300b. In some embodiments, a cable is used that has connectors at both ends thereof, wherein one of the connectors is configured to connect to a corresponding connector of a sleeve device and the other connector is configured to connect to a corresponding connector of a controller device.

### Hypothermia Risk Determination

In some implementations, the present disclosure relates to systems, devices, and methods for combining risk assessment/determination for patient hypothermia with a temperature management/therapy sleeve to enable automated regulation of patient core body temperature and prevention of hypothermia may include. Such systems/devices may include, for example, control circuitry configured to operate and/or generate heating and/or compression control signals based on and/or in response to one or more of: temperature readings/data (e.g., set(s) of temperature-relevant inputs); hypothermia risk determinations or parameters (e.g., from a risk-weighted, self-adjusting computation process for determining a patient's risk for developing hypothermia); and certain control logic (e.g., proportional-integral-derivative- (PID) derived control algorithm(s) configured to integrate with the heat and/or intermittent compression elements of the temperature-management sleeve/device).

Various inputs and/or datatypes may be utilized in controlling a patient's temperature to avoid hypothermia. For example, in some embodiments, temperature control may involve generating step function control signals to adjust temperature for patient warming. Through use of a risk-weighted/based computation/calculation process for controlling temperature, embodiments described herein may allow users to set a temperature management device and the device may be configured to automatically manage various patient-warming devices based on a variety of risk-related data structures/signals with or without additional user input.

In some embodiments, a patient's core temperature may be estimated or determined based at least in part on surface temperatures of the patient and/or ambient temperatures, such as may be determined based on signals from the sensor(s) 16 shown in FIG. 1. Such measured temperature(s) may provide an indication of how the patient's temperature may change over time. For example, surface temperatures may be utilized in determining, by control circuitry, a time value parameter indicating how long a period of time is expected until a patient may be in a range of hypothermia given current (e.g., sensor-based) conditions.

Additional parameters on which temperature-management signals may be based include parameters related to administration of anesthesia. When anesthesia is administered, a patient's brain may lose the ability to manage its body temperature to some degree. For example, in some situations, an anesthetized patient may experience dilated blood vessels even when the patient's body temperature is relatively low. When the patient's heart then pumps relatively cold blood from the patient's extremities, the patient's core temperature can be further lowered. In some embodiments, a clinician may provide input to a system (e.g., using the user I/O component(s) 52 shown in FIG. 1 and described above) indicating when anesthesia is administered. In some embodiments, a system may be configured to automatically determine that anesthesia has been administered. For example, given that heart rate generally drops with anesthesia, in some embodiments, a temperature-management system may be configured to determine that anesthesia has been administered automatically when it is determined (and/or in response to such determination) that a patient's heart rate has dropped below a predetermined threshold level or by a predetermined amount. Various inputs may be utilized in predicting a patient's future body temperature.

Determining a patient's risk for developing hypothermia may be based at least in part on various primary and/or secondary inputs/parameters (e.g., generated and/or stored parameter values, flags, or the like). Characterization as primary and secondary inputs/parameters can be further segmented/parsed as metadata types and/or data received and sent to sensors. In some embodiments, data from primary inputs may be utilized by the system for effective hypothermia prediction and prevention. That is, as used herein, “primary inputs” may refer to inputs that, according to some embodiments, are used to determine temperature control signals for managing patient temperature.

In some embodiments, certain data inputs/parameters used to monitor a patient and/or dynamically manage temperature conditions for the patient are illustrated in FIG. 4, which provides an example risk-weighted self-adjusting temperature-management process 400. The process 400 may be implemented in whole or in part by certain control circuitry of a temperature-management system, such as by control circuitry associated with one or both of a temperature-management controller and a temperature-management device (e.g., wearable sleeve device), as may be similar in certain respects to corresponding components in the system 10 of FIG. 1, described in detail above. In some embodiments, the process 400 can be implemented to determine a patient's hypothermia risk. Such data inputs/parameters may include, for example, static inputs 402 (e.g., procedure data 410, including procedure type and/or length) and continuous and/or time-varying inputs/parameters 412 from sensors, which may include, for example, stored and/or generated values indicating time-to-induction (e.g., of anesthesia) and/or peripheral temperature readings 416 (e.g., supplied by the control circuitry of the temperature-management system).

Certain types of parameter data/values may improve the accuracy of hypothermia risk determination while not being necessary for hypothermia risk determination. Such inputs/parameters may be referred to as “secondary” inputs/parameters. Although referred to below as “secondary” inputs/parameters, it should be understood that such parameters and/or associated values may be of any suitable or desirable type. In some implementations, the availability and/or inclusion of such secondary inputs may improve the accuracy of the calculation and/or temperature-management process 400, and by extension, the efficacy of prevention of hypothermia. Secondary static inputs 402 (e.g., metadata) may be sampled or determined/recorded at least once, such as prior to the relevant medical operation or during another period, and/or may not be sampled intraoperatively. Secondary static inputs 402 may include, for example, demographic data 404 (e.g., age, body mass index (BMI), and/or sex of the patient), comorbidity data 406 (e.g., American Society of Anesthesiologists (AS) grade and/or any of various risk factors including cancer and/or other disease risk, patient smoking habits, etc.), pharmacological agents 408 (e.g., premedication, anesthesia, and/or analgesia), procedure/timing-related data 410, and/or the like.

Secondary time-varying/dynamic parameter/input data 412 may be provided by and/or determined based on signals generated by sensors that may be a part of a temperature-management system. Secondary time-varying/dynamic parameter/input data 412 may include, for example, peripheral temperature 416 readings and/or environmental information 418 (e.g., temperature of the post-anesthesia care unit (PACU) and/or operating room, etc.). In some implementations, data collected in real time by one or more monitor devices and/or associated sensor(s) (e.g., a Philips anesthesia monitor) may be accessed intermittently, sporadically, periodically, on a delayed basis, and/or intraoperatively, wherein such data may serve as a basis for temperature management and/or hypothermia risk determination by system control circuitry. Types of time-varying data that may be used by control circuitry for temperature control and/or hypothermia risk determination may include, for example, core (and/or peripheral) temperature readings 414 (e.g., current value, rate of change, etc.), non-temperature vital signals 420 (e.g., heart rate, blood pressure, carbon dioxide level/values, oxygen level/values, and/or respiratory rate), and environmental information 418 (e.g., room temperature, use of heating measures, under-warming blanket, and/or intravenous line).

One or more parameters/inputs used in temperature-management process in accordance with aspects of the present disclosure may be assigned a risk weight 401. For example, a risk weight 401 may indicate how significant a given parameter/input may be in determining a patient's total risk of hypothermia. For example, while a patient's core temperature 414 and demographic information 404 (e.g., age) may both be parameters/inputs used in determining the patient's risk of hypothermia, the core temperature 414 of the patient may be relatively more determinative of risk than certain of the demographic information 404 and may accordingly be assigned a higher weighting. In some embodiments, a risk weight 401 may be time-varying. For example, the onset of anesthesia may be weighted with relatively greater risk of causing hypothermia immediately following administration of the anesthesia in comparison to a relatively lower risk towards the end of a surgical procedure. Parameter-weight correspondence information may be stored in one or more data storage devices of the system and utilized by control circuitry to drive temperature management control signal generation and/or provision. In some embodiments, the one or more data storage devices may be configured to store personalized and/or otherwise associated risk profiles. For example, a patient-specific risk profile identifying particular risk weight values and/or risk factors may be associated with a particular patient.

The process 400 may involve one or more operations relating to determination of one or more value-to-risk transformations/determinations 422. For example, value-to-risk transformation/determination 422 may involve accessing stored data (e.g., a lookup chart or other data structure(s)/type(s) stored in non-volatile or volatile data storage of the temperature-management system) to correlate measured parameter/input data to stored risk data. In some embodiments, risk data may provide a value between 0 and 1 to indicate how predictive/determinative each input may be of hypothermia risk and/or other issue(s).

In some embodiments, the temperature-management process 400 may further involve a relative-risk determination/transformation 424, which may be based at least in part on risk weight data 401 to indicate the relative risks of each parameter/input value relative to one or more other parameters/inputs. The relative-risk transformation 424 may be based at least in part on one or more of the static and/or dynamic parameters/inputs associated with the process 400. For example, demographic data 404 may be associated with a value-to-risk transformation 422 value of 0.7 (i.e., a score of 7 out of 10, with 10 being the highest risk of hypothermia). If demographic data 104 is the only parameter/input on which hypothermia risk determination is based, the patient may be determined to be associated with a risk value 426 of 0.7. In other words, demographic data 404 may be wholly determinative of the risk value 426 if demographic data 404 is the only parameter/input considered (or another parameter if such parameter is the only parameter considered). However, if other parameters/inputs are considered that have, for example, a relatively higher weighting than demographic data 404, the demographic data 404 may have a relatively low effect on the risk value 426. The risk value 426 may represent various determinations/calculations which may be performed based on any of the various parameter/input and/or transformations in the process 400.

In some embodiments, the process 400 may involve determining various derived parameters/inputs 428. Derived parameters/inputs 428 may include various computations to indicate how a patient's temperature may change over a period of time. In some embodiments, derived parameters/inputs 428 may be determined based at least in part on past measurement(s) (including, e.g., noise-filtered signals 430 indicative of patient temperature values) and/or summary statistics 432.

Static parameters/inputs 402 and/or continuous parameters/inputs 412 may be utilized in the risk calculation 426. Static parameters/inputs 402 may be utilized with respect to hypothermia risk determination 426 prior to onset of temperature management/therapy. In some embodiments, parameters/inputs may be input/entered by a user via manual entry (e.g., by clinical staff) and/or electronically/automatically through integration with data records (e.g., patient health record (PHR) systems and/or 3rd party data-integration vendor(s) of said data records).

Dynamic parameters/inputs 412 may be provided by various devices of the temperature-management system (e.g., sleeve(s)) and/or from other sources. The system may be configured to collect peripheral temperature 416, environmental temperature 418, and/or core temperature 414 data. Vital sign data 420 and/or other external data may be collected from various data records, for example.

In some embodiments, various dynamic parameters/inputs 412 may be pre-processed by the system in order to generate noise-filtered signals 430 and/or summary statistics 432. Noise-filtered signals 430 may eliminate signal artifacts (e.g., to provide noise smoothing). Summary statistics 432 may comprise aggregated statistics of various measurements (e.g., baseline, rate of change, future value prediction) that may be required or desired/helpful for the risk value 426 determination. Summarizing statistics 432 can include, for example, signal noise smoothing (e.g., filtering to remove noise artifacts from a signal), signal baseline (e.g., average of signals over time), rate-of-change estimations (e.g., derivative of the signal over time), and/or value predictions (e.g., use rate-of-change to project future state/temperature).

A patient temperature prediction 434 may be determined based at least in part on one or more derived parameters/inputs 428. In some embodiments, the risk value 426 may be based at least in part on the temperature prediction 434 and/or one or more user- and/or system-specific predictions 426 indicating how long until the patient may reach the predicted temperature, which may be specified in minutes or any other unit of time. The risk calculation 426, temperature prediction 434, and/or time prediction 426 may be displayed in a display 438 and/or may be used be a controller to adjust and/or maintain heating at one or more sleeves administered to a patient.

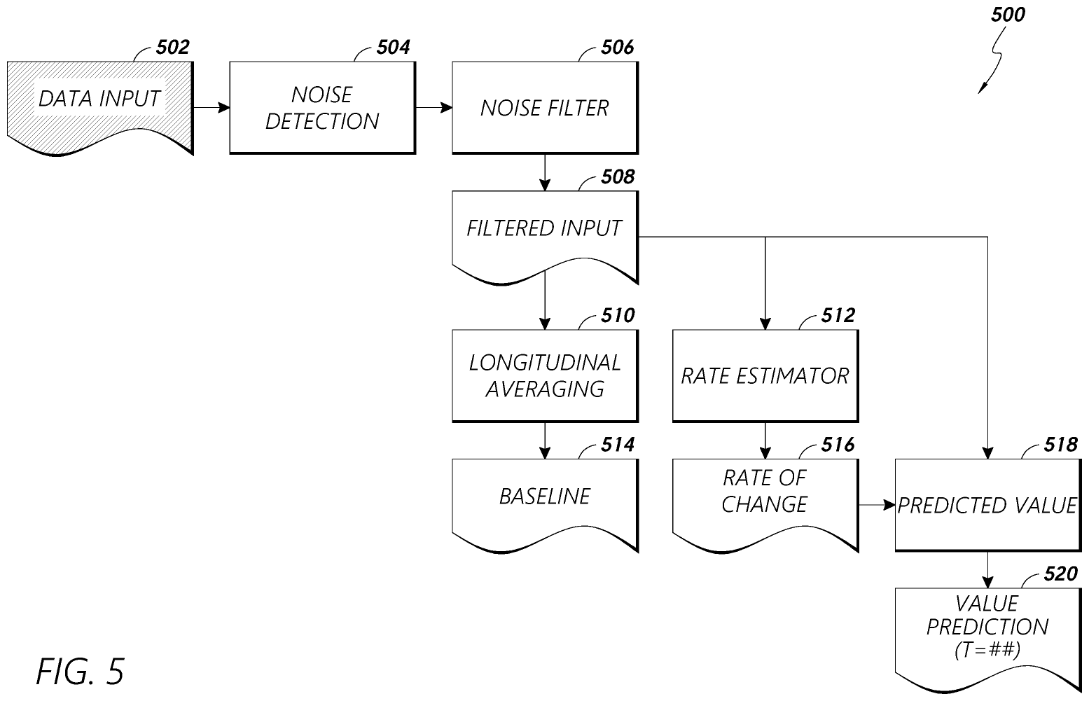

FIG. 5 illustrates a process 500 for predicting a patient's future body temperature value 520 given certain data input parameter(s) 502. The various functional modules and/or features relating to FIG. 5 and the process 500 can be performed at least in part by control circuitry of a temperature management device (e.g., wearable sleeve device) and/or a temperature management controller. Furthermore, the disclosed modules and features of FIG. 5 can represent implementation aspects relating to certain blocks of the process 400 of FIG. 4. The data input 502 may be a static input (e.g., a core temperature sample, as reported by various sensors such as esophageal, nasopharyngeal, bladder, tympanic, skin and/or other sensors) or a dynamic input. The process 500 may involve performing noise detection 504 and/or noise filtering 506 to generate a filtered input 508 (i.e., a noise-filtered signal). The filtered input 508 may represent a derived input and/or smoothed version of the data input 502. The process 500 may further involve generating rate-of-change value(s) 512 (e.g., in degC/min) and/or predicted temperature value(s) 518 (e.g., in Celsius) relating to a future time using the filtered input 508. In some embodiments, the rate-of-change value 516 may be computed through use of a rate-of-change estimator 512. The process 500 may further involve calculating a baseline metric 514 by evaluating a longitudinal averaging 510 over a period of time (e.g., a median temperature value as sampled over the last four hours). In some embodiments, the process 500 may involve generating secondary inputs (e.g., filtered inputs 508, rate-of-change values 516, baseline metrics 514, etc.) using various time-varying inputs/measurements including temperature, heart rate, oxygen concentration, and/or various vital signals.

Signal noise smoothing can be achieved through implementation of one or more filters (e.g., Kalman filter, or the like) applied to current and/or previous readings of a signal. The filter(s) may be configured to act as a recursive estimator which can compare the current (measured) value to the system's estimation (prediction) for the current value to identify and eliminate noise in the signal.

In some embodiments, a simple filter (e.g., a finite impulse response filter (FIR)) may be applied to various measurements. Filter coefficients may be designed to eliminate high frequency data from signals. A moving average (mean) filter may be utilized, in which a given number of measurements may be assigned the same weight in the filter.

The baseline 514 of a signal can represent a running average (mean) of the signal over a period of time (e.g., collected over the past 3 hours). In some embodiments, the baseline 514 can be computed on the smoothed signal to minimize influence of noise artifacts.

The rate-of-change 516 of a signal can represent the velocity of the signal over time. In some embodiments, the rate-of-change 516 can be computed from a rate estimator functional module 512 by comparing the current smoothed/filtered value 508 (x_k) to the next estimated value (x_klk). The formula may be the following:

dx_k/dt=[(x_klk)−(x_k)]/[sampling time] (1)

In some embodiments, the rate-of-change 516 may be computed through application of a Savitzky-Golay filter. The Savitzky-Golay algorithm applies an FIR to the most recent n-samples of data to estimate the derivative over the observed period of time (n samples). This computed derivative may be less reactive to rapid swings when compared to other estimators (e.g., a Kalman estimator).

The predicted value 518 for a signal can be computed by summation of the current smoothed/filtered signal 508 (x_k) and the product of the rate-of-change 516 (dx_k/dt) and the amount of time to project into the future (e.g., 30 min). For example, the prediction calculation 520 can be the following:

Prediction_k=x_k+(30*dx_k/dt) (2)

The filtered input 508, baseline 514, rate-of-change 516, and/or value prediction 520 may each represent derived inputs of the data input 502.

Risk transformations may represent conversions of real signals (e.g., temperature values) into a normalized risk value (e.g., 0-1). Examples of risk transformations can relate to the use of diagnostic tools like hospital scorecards in health care environments. Core temperature may be a direct risk input for hypothermia (by definition, hypothermia is defined by core temperature below 36° C.). In situations where the system has a dynamic/continuous reading of core temperature, both the current temperature and the trend in temperatures may have significant weight in the risk determination. For example, current temperature values below 36° C., between 36° C. and 37° C., and above 37° C. may correlate to risk transformation values of 10, 5, and 1, respectively. Rate-of-change 516 values in ° C./min of −0.01, −0.03, and −0.05 may correlate to risk transformation values of 1, 2, and 3, respectively, for example. Although certain risk values are disclosed herein, it should be understood that any types of risk values or scales may be implemented in embodiments of the present disclosure.

Peripheral patient temperature (e.g. skin surface temperature) may be an indirect predictor of hypothermia. Therefore, peripheral temperature values may serve as a non-zero-weighted parameter for hypothermia risk determination. For example, peripheral temperature may be associated with a risk value that is less than a risk value associated with core temperature. A similar risk index may be applied for any individual patient temperature readings. The individual risk values may be weighted relative to each other.

Risk-associated weighting of each parameter may be determined/translated based at least in part on pre-known clinically significant odds ratio when comparing patient populations. For example, patient ages of less than 15 years, between 15 and 64 years, and over 64 years (or any other age ranges) may be correlated with relative risk values of, for example, 1.00, 1.67, and 2.62, respectively, or any other values. American Society of Anesthesiologists (ASA) ratings of 1, 2, 3, 4, and 5 may correlate to relative risk values of, for example, 1, 1.8, 1.8, 3.2, and 19.9, respectively, or any other values. Body fat and/or body mass index (BMI) patient levels of n % may correlate to a relative risk value of 1+0.025*n, or any other relationship/values. Preoperative temperature values in C of less than 36 and greater than or equal to 36 may correlate to relative risk values of 1 and 0.3, respectively, or any other temperature ranges and/or risk values. Surgery magnitude designations of “minor,” “intermediate,” and “major” may correlate to relative risk values of 1, 5, and 10, respectively, or any other values or designations. Surgery duration values in hours of less than or equal to 2 and greater than 2 (or any other time periods) may correlate to relative risk values of 1.0 and 4.5, respectively, or any other values. Anesthesia types of regional, general, and combined (or any other type designations) may correlate to relative risk values of 0.22, 1, and 2.77, respectively, or any other values.

Various risk factors may be collected and determined/transformed from a measured (or derived) value (e.g., degC) to a risk metric with a value between 0 and 1. However, individual risk factors can generally have different impact on a patient's risk for hypothermia. For example, the environmental temperature (e.g., 25 C) may indicate a moderate risk (e.g., 0.75) for hypothermia, but relative impact of the environmental temperature may be small when compared to the patient's actual core body temperature (e.g., 36.7 C, translating to a risk metric of 0.3, for example). In this case, the relative weight of environmental temperature may be much smaller than the core body temperature reading. The relative risk may be calculated as a product of a given risk metric and the relative weight of the given risk metric.

The relative weights assigned to each metric may be dynamically configurable and/or may change depending on a number of factors. For example, the weights may be modified based on static inputs, such as patient demographic and operating mode (e.g., pre-op vs. intra-op vs. post-op) data. For example, an older patient with elevated CVS risk (e.g., due to smoking) could have a different set of weights applied as compared to a 20-year old. patient with no additional demographic risk factors. Additional factors may include the quantity of anesthesia.

In some embodiments, risk index weights may shift based on operating mode as well as with patient/procedure demographic information. For example, a patient with cardiovascular system complications may have elevated blood pressure. The contribution of the cardiovascular system and/or blood pressure complications towards hypothermia risk in the patient may be lower than for a patient who has no heart disease and/or nominally normal blood pressure.

Some embodiments may involve performing a weighted and/or normalized summation on some or all available risk metrics. Relative weights may be preconfigured and/or may be modified based on hospital protocol, procedure type, and/or physician decision. An overall risk value for a patient may be calculated by dividing a summation of all relative risk values for a given metric by a summation of all metric-specific coefficients/weights using a relative weighting. The resulting overall risk value may be a value between 0-1 and/or may reflect the system's determination of a patient's risk for hypothermia. Such determination may be generated/performed at least in part by control circuitry of the temperature-management system as described herein.

Patient peripheral (e.g., limb) temperatures may be expected to be lower than the core body temperature. Some embodiments may involve implementing a model that accounts at least in part for the transfer of heat through lower limb tissue and vasculature to translate measured peripheral temperatures into estimates of tissue temperature (e.g., by depth) and/or core body temperature.

Estimated core body temperature may be used in place of direct core temperature measurements when direct core temperature measurements may not be available. Furthermore, actual and/or estimated tissue temperatures may be used by the system to monitor patient burn risk, particularly in situations where an external heat source is applied to a peripheral limb. For example, tissue temperature data may be generated and/or provided by one or more temperature sensors (e.g., thermistors) integrated with a wearable sleeve device in accordance with aspects of the present disclosure.

In some embodiments, a patient's future core temperature may be predicted approximately thirty minutes, or other amount of time, in advance using one or more of the following parameters: the patient's current temperature, a temperature rate-of-change, and/or anesthesia depth. For example, the future temperature may be determined based at least in part on a sum of the current temperature, the temperature rate-of-change, and an anesthesia modifier factoring in the concentration of anesthesia.

In some embodiments, outputs of a risk value determination may be used as inputs for a controller (e.g., the controller 50 of FIG. 1) to adjust the amount of heat transferred into the patient's body. In some implementations, heat transfer may be dually controlled by adjusting the temperature of heating element(s)/mechanism(s) associated with a wearable sleeve device disposed on the patient and/or the rate of venous return of implemented by compression element(s)/mechanism(s) associated with the sleeve device (e.g., adjusting rate-of-flow and/or inflation period/cycle for inflatable bladder compression). In certain situations, the temperature-management controller may be configured to control heating and compression elements together, treating them as a single mechanism/transducer. For example, at therapy initiation (e.g., device start), the device may rapidly reach the target temperatures and pressure.

The temperature-management controller may be configured to operate each heating element (e.g., 2 per limb for each of the sole of the food and the back of the knee/popliteal fossa) independently while controlling the compression elements (e.g., 1 per limb for a calf portion of the sleeve device) together. Independent heating element control may advantageously allow for relatively finer tuning of heat transfer to the body. In some embodiments, a temperature-management controller may be configured to alternate heating between, for example, foot and popliteal fossa locations to support higher device temperatures (e.g., increased heat transfer) without increasing tissue burn risk.

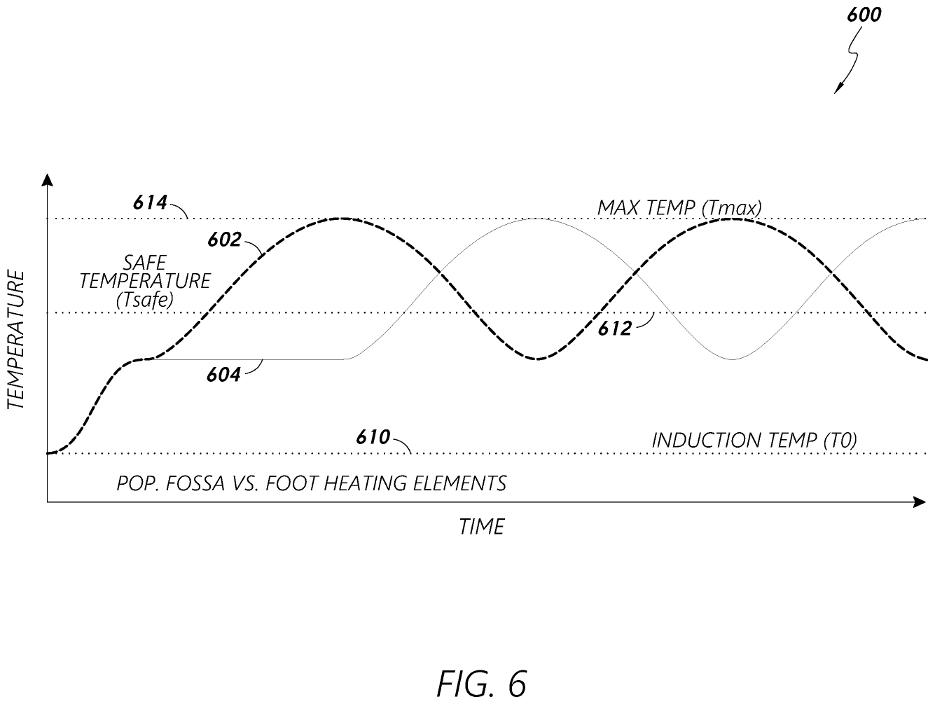

FIG. 6 provides a graph 600 illustrating temperature values at different moments of time. A first line 602 of the graph 600 may represent temperature values at a first target area of a patient (e.g., the popliteal fossa) and a second line 604 may represent temperature values at a second target area (e.g., the sole of the foot). Temperature values may vary between an induction temperature value 610 and a maximum temperature value 614. A certain temperature value, or range of temperature values, between the induction temperature value 610 and the maximum temperature value 614 may be considered safe and/or desired temperature value(s) 612. In some embodiments, the maximum temperature value 614 may correspond to a temperature at which there may be a risk of burning at the target area. For example, the maximum temperature 614 may represent a temperature beyond which the risk of burn is greater than a predetermined threshold. As temperature at one of the target areas increases to above the safe temperature value 612 and/or at or near the maximum temperature value 614, the temperature may be decreased to prevent burning. Similarly, as the temperature drops below the safe temperature value 612, the temperature may be increased. That is, embodiments of the present disclosure provide for the intermittent heating of the sole of the foot and the popliteal fossa in a back-and-forth manner to provide improved heating while operating within safe temperature ranges. For example, the heating element(s) associated with the foot and the popliteal fossa may be operated in an at least partially alternating manner, wherein the maximum heating level for the temperature-management process/system is not implemented for one of the foot and popliteal fossa during a time in which the maximum heating level is implemented for the other. In some embodiments, temperature may be controlled manually and/or at least partially automatically/electronically using the temperature-management controller/control circuitry.

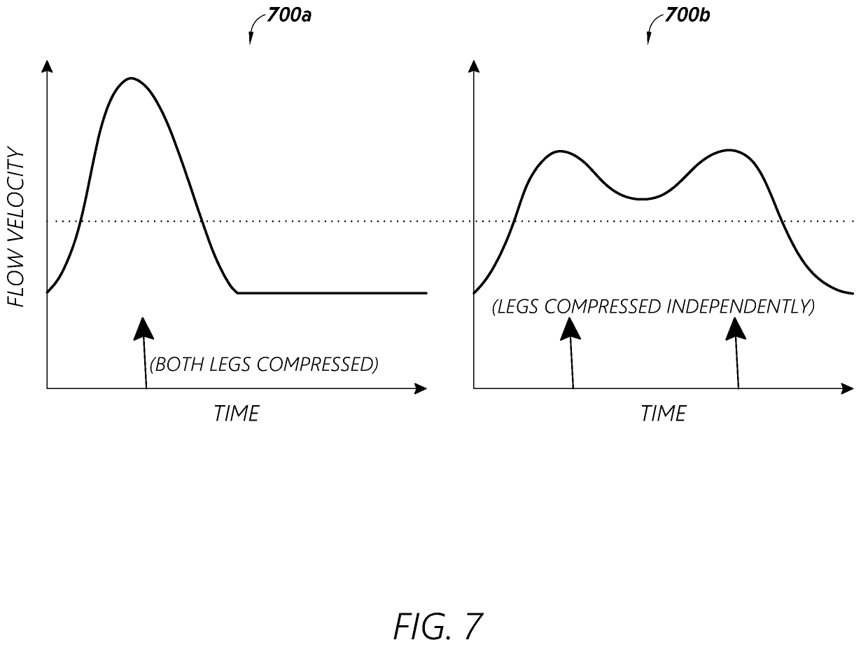

In some embodiments, a temperature-management controller may be configured to control each heating element (e.g., 2 per limb) and/or compression element (e.g., 1 per limb) independently. FIG. 7 provides graphs illustrating venous flow velocity over time for multiple-limb temperature management solutions. In some embodiments, a temperature-management system may comprise multiple sleeves/sleeve components (e.g., each at a different limb of a patient) and/or a sleeve configured to compress multiple limbs of the patient. A first graph 700a represents flow velocity values for a system configured to compress multiple (e.g., two) limbs of a patient together (i.e., using the same control signals for both sleeve devices). As shown in FIG. 7, the flow velocity when both limbs are compressed may spike and drop dramatically. The second graph 700b illustrates venous flow velocity for a system in which multiple limbs are compressed independently of each other. For example, each sleeve of multiple sleeves may be controlled independently to provide enhanced therapy associated with venous return. As shown in the second graph 700b, venous flow velocity values may reduce slightly after an initial spike associated with compression of one limb, but the velocity values may be maintained more effectively than in the system shown in the first graph 700a as they rise again in connection with compression of the other limb. In some embodiments, a system may be configured to alternate compression between each leg of a patient to improve the venous return profile.

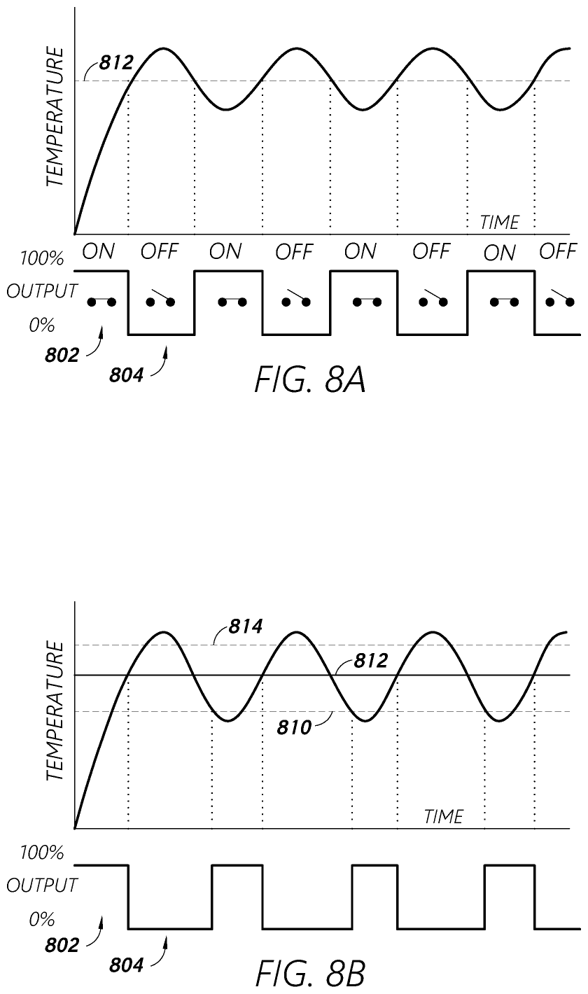

FIGS. 8A and 8B illustrate on-off cycles performed at a temperature-management controller for one or more sleeves configured to warm and/or compress one or more target areas of a patient's body. In some embodiments, the controller may utilize a process (e.g., a damped proportional-integral-derivative (PID) control algorithm) to control core body temperature. The process may be designed to minimize overshoot and/or undershoot issues of a simple treat-to-target and/or treat-to-range algorithm. As shown in FIGS. 8A and 8B, a controller may be configured to enter an ON state 802 when temperature is above a safe temperature value 812 and/or at or near a maximum temperature value 814. Similarly, the controller may be configured to enter an OFF state 804 when the temperature is below the safe temperature value 812 and/or at or near a minimum temperature value 810. The pulse width of the ON-cycle relative to the OFF-cycle can determine the amount heating and/or blood flow augmentation implemented. In some embodiments, the controller may be configured to apply heat when the patient is hypothermic and/or predicted to become hypothermic based at least in part on various calculation processes described herein.

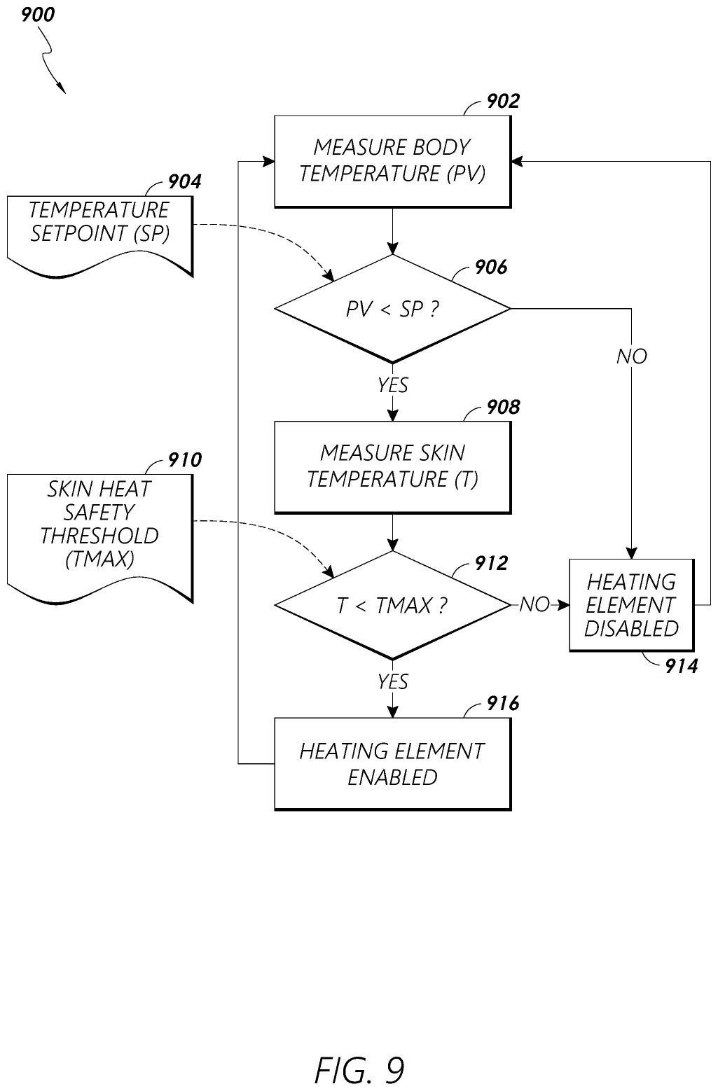

FIG. 9 provides a process 900 for enabling/activating heating elements at sleeve devices providing heat to a patient. The process 900 may be implemented in whole or in part by certain control circuitry of a temperature-management system, such as by control circuitry associated with one or both of a temperature-management controller and a temperature-management device (e.g., wearable sleeve device), as may be similar in certain respects to corresponding components in the system 10 of FIG. 1, described in detail above.

At block 902, the process 900 involves measuring and/or estimating a present value (PV) of the patient's body temperature. For example, the measured/estimated temperature may be directly-measured core temperature or may be estimated temperature based on measured peripheral (e.g., skin) temperature. In some embodiments, the temperature PV may be measured using one or more sensors attached to and/or otherwise used in conjunction with a sleeve administered to, or otherwise disposed on, the patient.

At block 904, the process 900 involves determining a temperature set point (SP) (e.g., target temperature value). In some embodiments, different areas of a patient's body may have different SP values. The temperature SP may be based on a predetermined temperature level associated with a burn risk above a certain threshold. The temperature SP may represent a body/core temperature of the patient.

At decision block 906, the process 900 involves determining whether the PV of a given area of the patient's body is lower than the SP value relevant for that area. If the PV is lower than the SP value, the process 900 continues to block 908. If the PV is equal to or greater than the SP value, the process 900 continues to block 914.

At block 908, the process 900 involves measuring the patient's skin temperature (T). The skin temperature T may be measured to determine a risk of burning at the patient's skin. At block 910, the process 900 involves determining a skin heat safety threshold (TMAX), wherein the determination at block 912 may be based at least in part on the threshold TMAX. TMAX may be indicative of a temperature at which the patient may be at risk of localized burning.

At decision block 912, the process 900 involves determining whether T is less than TMAX. If T is less than TMAX, the process 900 continues to block 916. If T is equal to or greater than TMAX, the process 900 continues to block 914.

At block 914, the process 900 involves deactivating or otherwise disabling or throttling one or more heating element at the given area of the patient's body. At block 916, the process 900 involves enabling and/or increasing activity of the heating element(s) (e.g., increasing the duty cycle).

An applied potential and/or adjustment in duty cycle of a heating element at a sleeve may be modulated based on proportional, integral (Ki), and/or derivative adjustments in response to measured error in temperature as compared to the target temperature. In some embodiments, the further away from the target temperature, the more power is applied to the heating element(s) and/or compression element(s). An integral component may allow for correction of an offset error. A derivative component may be useful in reducing a transient time effect (e.g., overshoot).

In some embodiments, a temperature-management controller may be configured to use individually-actuated heating pads and/or a variety of threshold values (e.g., safe and/or maximum temperature values). The controller may be configured to individually control each heating element based at least in part on each respective threshold value.

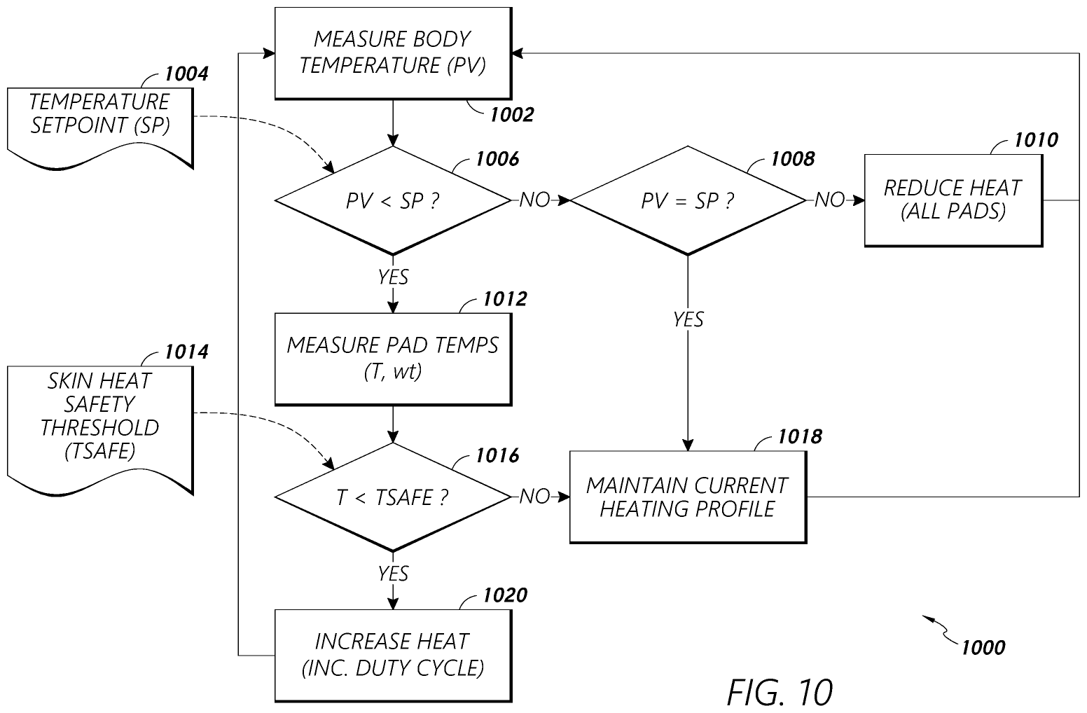

FIG. 10 provides a process 1000 for increasing temperature at heating elements of sleeve devices providing heat to a patient in accordance with aspects of the present disclosure. The process 1000 may be implemented in whole or in part by certain control circuitry of a temperature-management system, such as by control circuitry associated with one or both of a temperature-management controller and a temperature-management device (e.g., wearable sleeve device), as may be similar in certain respects to corresponding components in the system 10 of FIG. 1, described in detail above.

At block 1002, the process 1000 involves measuring and/or estimating a present value (PV) of the patient's body temperature. For example, the measured/estimated temperature may be directly-measured core temperature or may be estimated temperature based on measured peripheral (e.g., skin) temperature. In some embodiments, the temperature PV may be measured using one or more sensors attached to and/or otherwise used in conjunction with a sleeve administered to, or otherwise disposed on, the patient.

At block 1004, the process 1000 involves determining a temperature set point (SP) (e.g., target temperature value). In some embodiments, different areas of a patient's body may have different SP values. The temperature SP may be based on a predetermined temperature level associated with a burn risk above a certain threshold. The temperature SP may represent a body/core temperature of the patient.

At decision block 1006, the process 1000 involves determining whether the PV of a given area of the patient's body is lower than the SP value relevant for that area. If the PV is lower than the SP value, the process 1000 continues to block 1012. If the PV is equal to or greater than the SP value, the process 1000 continues to block 1008.

At block 1008, the process 1000 involves determining whether PV is equal to the SP value. If PV is equal to SP, the process 1000 continues to block 1018. If PV is not equal to SP, the process 1000 continues to block 1010.

At block 1010, the process 1000 involves reducing heat at one or more heating elements (e.g., pads) associated with one or more sleeves administered to, or otherwise disposed on, the patient.

At block 1012, the process 1000 involves measuring temperatures (T) at heating elements (e.g., pads). For example, heating elements may have temperature sensor(s) (e.g., thermistor(s)) associated therewith. At block 1014, the process 1000 involves determining a skin heat safety threshold value (TSAFE). At decision block 1016, the process 1000 involves determining whether T is less than TSAFE. If T is less than TSAFE, the process 1000 continues to block 1020. If T is greater than or equal to TSAFE, the process 1000 continues to block 1018.

At block 1018, the process 1000 involves maintaining the current heating profile at one or more heating elements. At block 1020, the process 1000 involves increasing heat (e.g., increasing the duty cycle) at one or more heating elements.

In some embodiments, a temperature-management controller may treat multiple heating elements as a single element/transducer. For example, the controller may be configured to set the heating elements to a single common temperature and/or drive the heating elements using common or similar control signals. However, in some cases, one or more heating elements may reach the skin heat safety threshold (TSAFE) while the core body temperature remains below the set point. In such cases, the controller may be configured to increase heating element temperatures from TSAFE to a higher maximum temperature (TMAX) for a limited duration. In some cases, at least some areas of tissue may be heated to TMAX for a period of time without burning. Accordingly, temperature at one or more heating elements may be cycled higher and lower. The controller may be configured to alternate heating and non-heating (e.g., cooling) between TSAFE and TMAX as between heating elements at different limbs to maintain elevated blood temperature in each limb. In such embodiments, a first heating element (e.g., at the popliteal fossa of the left leg) may be heated towards TMAX while a second heating element (e.g., at the foot of the left leg) may be heat-throttled/cooled towards TSAFE.

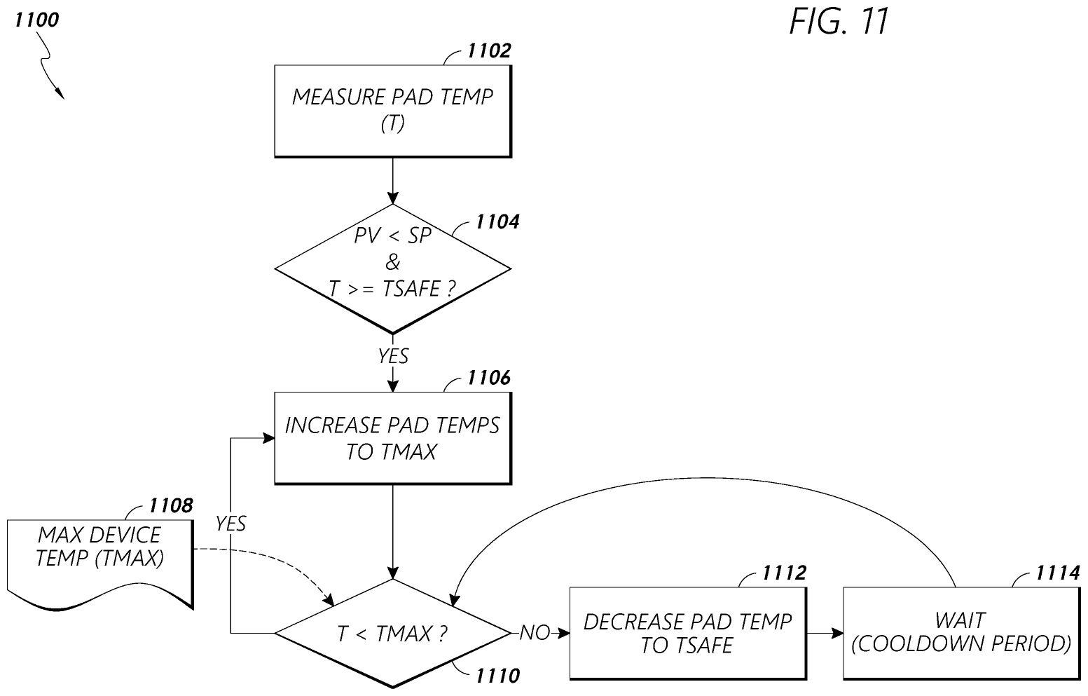

In some embodiments, activation/heating of different heating elements may be offset in time (e.g. by one cooldown period) in order to achieve a desired alternating heat profile. FIG. 11 illustrates a process 1100 for controlling a heating element of a sleeve in accordance with one or more embodiment of the present disclosure. The process 1100 may be implemented in whole or in part by certain control circuitry of a temperature-management system, such as by control circuitry associated with one or both of a temperature-management controller and a temperature-management device (e.g., wearable sleeve device), as may be similar in certain respects to corresponding components in the system 10 of FIG. 1, described in detail above.

At block 1102, the process 1100 involves measuring a heating element (e.g., pad) temperature (T). At decision block 1104, the process 1100 may involve determining whether a present measured and/or estimated value (PV) of a patient's body/core temperature is lower than a set point temperature value (SP) and whether T is greater than or equal to a skin heat safety threshold value (TSAFE). If PV is less than SP and T is greater than or equal to TSAFE, the process 1100 continues to block 1106.

At block 1106, the process 1100 involves increasing T to a maximum temperature value (TMAX), which may have previously been determined in connection with the operation(s) associated with block 1108. At decision block 1110, the process 1100 involves determining whether T is less than TMAX. If T is not less than TMAX, the process 1100 proceeds to block 1112.

At block 1112, the process 1100 involves decreasing T to TSAFE in some manner, such as by throttling/deactivating the heating element/pad associated with the temperature T. At block 1114, the process 1100 involves waiting for a period (e.g., one cooldown period), which may be any suitable or desirable period of time that is sufficient for the temperature T of the heating element(s) to trop below TMAX.

A temperature-management controller may be configured to operate within safe limits for temperature applied to skin tissue to avoid burns. Generally, for reference, certain human tissue may start to burn at temperatures above approximately 43° C. In some embodiments, tissue burn monitoring may be achieved through peripheral temperature probes placed between heating elements and patient skin. Peripheral temperatures may be translated into estimated tissue temperature using a physiological heat transfer model that can account for heat transfer through both tissue and heating sleeve materials.

The temperature-management controller can be designed with safety considerations in place to limit heating element temperature based on “heat capacity” of the surrounding tissue. For example, the controller may evaluate historical pad temperatures to monitor precisely the amount of time skin temperature has exceeded the safe threshold (e.g., 43° C.) and may adjust TMAX and/or cooldown periods accordingly. Various safety measures may include visual/audible alerts and/or warnings generated and/or provided by a controller in response to detected risks of, for example, tissue burning and/or hypothermia. Such safety measures may be configured to prompt clinicians to take particular actions to correct detected errors. For example, a safety measure may prompt a clinician to check a device connection and/or sleeve placement alignment, etc.

Deep vein thrombosis (DVT) prophylaxis can operate through sequential compression of the calf to increase circulation of blood throughout the body. Changes to the compression sequence may be implemented to modify the rate of blood flow. In some embodiments, venous return rate may be maintained at a sufficiently elevated level to prevent DVT. Heat transfer into tissue (and/or blood in underlying vessels) may occur on a comparable (or faster) time scale to the rate of compression. Venous return rate adjustments can affect the amount of heat that may be returned to the body's core by one or more sleeves.

Some embodiments may involve using independently controlled heating elements with adjustments for compression. For example, sleeve pressure ratings and/or compression frequency may be controlled in conjunction with heating at one or more sleeves.

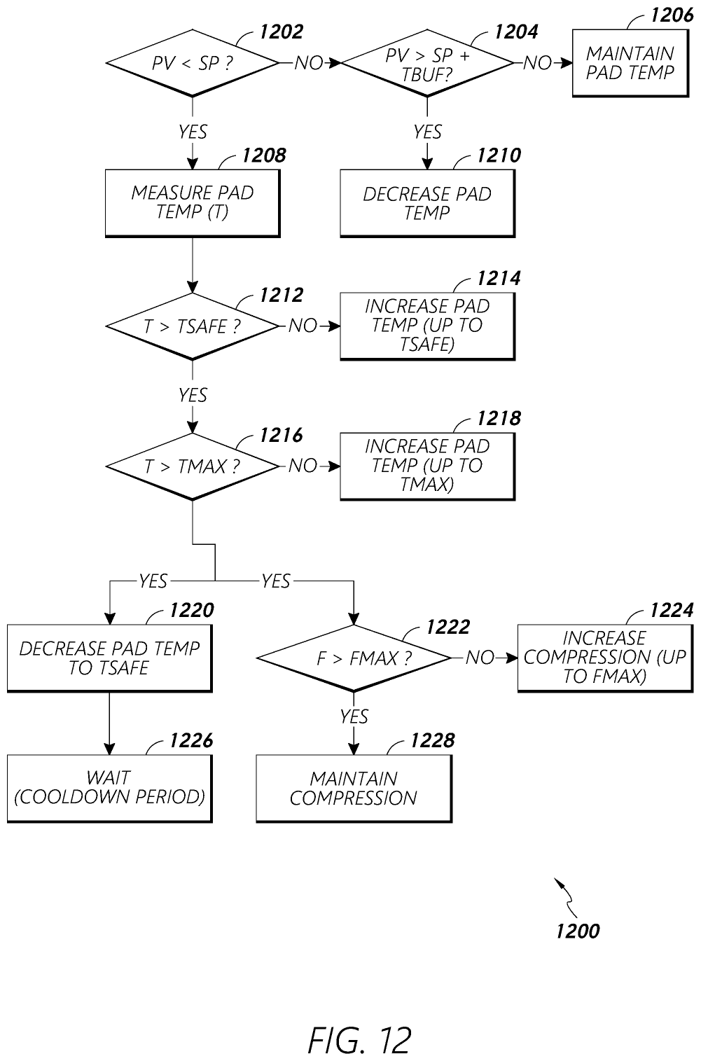

FIG. 12 provides a process 1200 for increasing and/or maintaining patient temperature using a sleeve configured to provide heat and/or compression to a patient. The process 1200 may be implemented in whole or in part by certain control circuitry of a temperature-management system, such as by control circuitry associated with one or both of a temperature-management controller and a temperature-management device (e.g., wearable sleeve device), as may be similar in certain respects to corresponding components in the system 10 of FIG. 1, described in detail above.

At decision block 1202, the process 1200 involves determining whether a present value of a patient's body temperature (PV) is less than a set point temperature value (SP). If PV is less that SP, the process 1200 proceeds to block 1208. If PV is greater than or equal to SP, the process 1200 proceeds to block 1204.

At decision block 1204, the process 1200 involves determining whether PV is greater than a sum of SP and a buffer value (TBUF). TBUF (e.g., −1° C.) may be configured to prevent rapid oscillation in heat output around SP. If PV is greater than the sum, the process 1200 proceeds to block 1210. If PV is not greater than the sum, the process 1200 proceeds to block 1206. At block 1206, the process 1200 involves maintaining temperature at one or more heating elements (e.g., pads).

At block 1208, the process 1200 involves measuring a temperature at one or more heating elements (T). At block 1210, the process 1200 involves decreasing the temperature at one or more heating elements.

At decision block 1212, the process 1200 involves determining whether T is greater than a safe heating element temperature threshold (TSAFE) to avoid burning tissue. If T is greater than TSAFE, the process 1200 proceeds to decision block 1216. If T is not greater than TSAFE, the process 1200 proceeds to block 1214. At block 1214, the process 1200 involves increasing T up to TSAFE.

At decision block 1216, the process 1200 involves determining whether T is greater than a maximum heating element temperature (TMAX) that is greater than TSAFE. Heat application between TSAFE and TMAX may be cycled to prevent tissue burning. If T is greater than TMAX, the process 1200 proceeds to block 1220 and decision block 1222. If T is not greater than TMAX, the process 1200 proceeds to block 1218. At block 1218, the process 1200 involves increasing T to TMAX. At block 1220, the process 1200 involves decreasing T to TSAFE. At block 1226, the process 1200 involves waiting a period (e.g., one cooldown period).

In some embodiments, steps of the process 1200 may be performed iteratively and/or cyclically. For example, after one or more heating elements are activated to increase T at blocks 1214 and/or 1218, the process 1200 may start over at decision block 1202 after a given period of time. In some embodiments, the number of times the process 1200 is repeated in which T is increased may indicate a safety concern and/or may cause activation of an alert/warning. For example, if T is increased for a particular amount of time, an alert at a controller may be activated to indicate to a clinician that the patient's body temperature is not increasing despite the activation of heating elements. Failure to increase the patient's body temperature may indicate failure of one or more heating elements and/or physiological issues of the patient.

At decision block 1222, the process 1200 involves determining whether a compression frequency (F) at one or more compression elements of the sleeve is greater than a maximum frequency of applied sequential compression (FMAX). If F is greater than FMAX, the process 1200 proceeds to block 1228. If F is not greater than FMAX, the process 1200 proceeds to block 1224. At block 1224, the process 1200 involves increasing F to FMAX. At block 1228, the process 1200 involves maintaining F.

In some embodiments, sleeve compression for DVT prophylaxis may have a range of acceptable pressure and frequency to achieve deep vessel collapse. Compression periodicity may oscillate (e.g., between 20 and 60 seconds). This range may be based at least in part on accepted clinical ranges for DVT prophylaxis therapy. The compression amplitude of a sleeve may be controlled by a compression chamber pressure. The applied pressure may range from 40-100 mmHg. Higher pressures may increase the peak blood flow velocity.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of assessing a patient's risk of hypothermia, the method comprising:
receiving a first input from a sleeve administered to a patient;
receiving a second input;
determining a first risk value based at least in part on the first input;
determining a second risk value based at least in part on the second input;
determining a first relative risk value of the first risk value based at least in part on comparing the first risk value to the second risk value;
determining a second relative risk value of the second risk value based at least in part on comparing the first risk value to the second risk value; and
generating a risk score for the patient.

2. The method of claim 1, wherein the first input is one of a group comprising core temperature data for the patient, peripheral temperature data, and vital signal data for the patient.

3. The method of claim 1, wherein the second input is one of a group comprising demographic data for the patient, comorbidity data for the patient, pharmalogical data for the patient, procedural data relating to a procedure involving the patient, core temperature data for the patient, peripheral temperature data, environmental data, and vital signal data for the patient.

4. The method of claim 1, further comprising:
assigning a first weight value to the first input; and
assigning a second weight value to the second input;
wherein determining the first relative risk value involves comparing the first weight value to the second weight value.

5. The method of claim 1, further comprising adjusting a temperature of the sleeve based at least in part on the risk score.

6. The method of claim 1, further comprising computing a rate of core temperature change value based at least in part on the first input.

7. The method of claim 6, further comprising determining a core temperature prediction for the patient based at least in part on the rate of core temperature change value.

8. The method of claim 7, further comprising adjusting a temperature of the sleeve based at least in part on the risk score.

9. A method comprising:
determining a set point core temperature value;
measuring a present core temperature value of a patient being treated with a sleeve comprising one or more heating elements;
comparing the set point core temperature value to the present core temperature value;
in response to determining that the present value is not less than the set point value, comparing the present value to a sum of the set point value and a buffer value;
in response to determining that the present value is not greater than the sum, maintaining a temperature setting at a first heating element of the sleeve; and
in response to determining that the present value is greater than the sum, decreasing the temperature setting at the first heating element of the sleeve.

10. The method of claim 9, further comprising:
in response to determining that the present value is less than the set point value, measuring a heating element temperature of the first heating element; and
comparing the heating element temperature to a safety threshold value.

11. The method of claim 10, further comprising, in response to determining that the heating element temperature is not greater than the safety threshold value, increasing the heating element temperature.

12. The method of claim 10, further comprising, in response to determining that the heating element temperature is greater than the safety threshold value, comparing the heating element temperature to a maximum temperature value.

13. The method of claim 12, further comprising, in response to determining that the heating element temperature is not greater than the maximum temperature value, increasing the heating element temperature.

14. The method of claim 12, further comprising, in response to determining that the heating element temperature is greater than the maximum temperature value, decreasing the heating element temperature.

15. The method of claim 12, further comprising, in response to determining that the heating element temperature is greater than the maximum temperature value, comparing a compression frequency of a first compression element at the sleeve to a maximum frequency value.

16. The method of claim 15, further comprising, in response to determining that the compression frequency is not greater than the maximum frequency value, increasing the compression frequency.

17. The method of claim 15, further comprising, in response to determining that the compression frequency is greater than the maximum frequency value, maintaining the compression frequency.
